(19) United States
(12) Patent Application Publication
(10) Pub. No.: US 2007/0149832 A1
(43) Pub. Date: Jun. 28, 2007

(54) PROCESS FOR THE PRODUCTION OF METHANOL FROM METHANE USING A METAL TRIFLUOROACETATE CATALYST

(76) Inventors: Wensheng Chen, Hoffman Estates, IL (US); Timothy A. Brandvold, Arlington Heights, IL (US); Maureen L. Bricker, Buffalo Grove, IL (US); Joseph A. Kocal, Glenview, IL (US); Joel T. Walenga, Lake Zurich, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/560,943

(22) Filed: Nov. 17, 2006

Related U.S. Application Data
(60) Provisional application No. 60/753,046, filed on Dec. 22, 2005.

(51) Int. Cl. C07C 29/03

(52) U.S. Cl. 568/910

(57) ABSTRACT

A process for converting methane to methanol using a homogeneous catalyst has been developed. The catalyst is a metal compound having an empirical formula of MₓXₘ where M is a metal such as Pd, Cu, Co, and Mn, X is an anion such as acetate, trifluoroacetate, sulfate, propionate, “m” is the oxidation state of M, and “x” is the anion valence of X. Generally the process involves contacting a gas stream containing methane with the homogeneous catalyst and an oxidant such as hydrogen peroxide at oxidation conditions to produce methyl trifluoroacetate. Finally, the methyl trifluoroacetate is hydrolyzed to give a methanol product stream.

### CROSS REFERENCE TO RELATED APPLICATION

This application claims priority from Provisional Application Ser. No. 60/753,046 filed Dec. 22, 2005, the contents of which are hereby incorporated by reference in its entirety.

### FIELD OF THE INVENTION

This invention relates to a process for converting methane to methanol using a homogeneous catalyst. The catalyst comprises a metal compound, where the metal is a transition metal such as palladium or copper, and the anion can be trifluoroacetate, acetate, propionate, etc. dissolved in a solvent such as trifluoroacetic acid. Generally the process involves contacting a gas stream containing methane with the homogeneous catalyst and an oxidant such as hydrogen peroxide at oxidation conditions to produce methyl trifluoroacetate. Finally, the methyl trifluoroacetate is hydrolyzed to yield a methanol product stream.

### BACKGROUND OF THE INVENTION

Today, both chemical and energy industries rely on petroleum as the principal source of carbon and energy. Methane is under-utilized as a chemical feedstock, despite being the primary constituent of natural gas, an abundant carbon resource. Factors limiting its use include the remote locations of known reserves, its relatively high transportation costs and its thermodynamic and kinetic stability. Methane's main industrial use is in the production of synthesis gas or syngas via steam reforming at high temperatures and pressures. Syngas in turn can be converted to methanol also at elevated temperatures and pressures. The production of methanol is important because methanol can be used to produce important chemicals such as olefins, formaldehyde, acetic acetate, acetate esters and polymer intermediates. The above two step process for the production of methanol is expensive and energy intensive with corresponding environmental impacts.

Selective oxidation of methane has been studied for over 30 years by individual, academic and government researchers with no commercial success. For example, Sen et al. in New J. Chem, 1989, 13, 755-760 disclose the use of Pd (O₂C Me)₂ in trifluoroacetic acid for the oxidation of methane to CF₃CO₂Me. The reaction is carried out for 4 days at a pressure of 5516-6895 kPa (800-1000 psi). E. D. Park et al. in Catalysis Communications, Vol. 2 (2001), 187-190, disclose a Pd/C plus Cu (CH₃COO)₂ catalyst system for the selective oxidation of methane using H₂/O₂. L. C. Kao et al. in J. Am. Chem. Soc., 113 (1991), 700-701 disclose the use of palladium compounds such as Pd (O₂CC₂H₅)₂ to oxidize methane to methanol in the presence of H₂O₂ and using trifluoroacetic acid as the solvent. U.S. Pat. No. 5,585,515 discloses the use of catalysts such as Cu(I) ions in trifluoroacetic acid to oxidize methane to methanol. WO 2004069784 A1 discloses a process for the oxidation of methane to methanol using transition metals such as cobalt or manganese in trifluoroacetic acid. Finally, M. N. Vargaftik et al in J. Chem. Soc., Chem. Commun. 1990(15) pp. 1049-1050 disclose results for a number of metal perfluoro acetate compounds. The metals which were found to be active were Pd, Mn, Co and Pb. Copper was found to have virtually no activity.

Applicants have developed a liquid phase process for the oxidation of methane to methanol. The process involves the use of a metal trifluoroacetate compound such as Pd (CF₃COO)₂ or Cu (CF₃COO)₂ dissolved in a solvent such as tri-fluoroacetic acid. This homogeneous catalyst is contacted with hydrogen peroxide and methane gas under mild conditions.

### SUMMARY OF THE INVENTION

As stated, this invention relates to a process for converting methane to methanol comprising contacting a gas stream comprising methane with a homogeneous catalyst and an oxidant selected from the group consisting of hydrogen peroxide, organic hydroperoxides and mixtures thereof at oxidation conditions to provide a methyl-ester and hydrolyzing the methyl ester at hydrolysis conditions to provide a methanol product stream; the homogeneous catalyst comprising a metal compound, having the empirical formula MₓXₘ where M is selected from the group consisting of palladium, copper, manganese, mercury, silver, cobalt, vanadium, platinum, lead, gold, niobium, chromium, molybdenum, tungsten, cerium and mixtures thereof, X is an anion selected from the group consisting of acetate, trifluoroacetate, sulfate, carbonate, halide, nitrate, perchlorate, propionate, pentafluoropropionate, acetylacetonate, and hexafluoroacetylacetonate, and “m” is the oxidation state of M, and “x” is the anion valence of X, dissolved in a solvent selected from the group consisting of trifluoroacetic acid, trifluoroacetic anhydride, pentafluoropropionic acid, acetic acid, supercritical carbon dioxide and mixtures thereof where the oxidation conditions comprise a temperature of about 25° C. to about 250° C., a pressure of about 103 kPa (15 psi) to about 6895 kPa (100 psi), a contact time of about 30 min. to about 24 hrs and an oxidant to M molar ratio of about 15 to about 15000.

This and other objects and embodiments will become clearer after a detailed description of the invention.

### DETAILED DESCRIPTION OF THE INVENTION

The present invention relates to a liquid phase process for the oxidation of methane to methanol. One necessary component of this process is a homogeneous catalyst which promotes the selective oxidation of methane. The catalyst comprises a metal compound dissolved in a solvent. The compounds have an empirical formula of MₓXₘ where M is selected from the group consisting of palladium, copper, manganese, mercury, silver, cobalt, vanadium, platinum, lead, gold, niobium, chromium, molybdenum, tungsten, cerium and mixtures thereof, X is an anion; examples of which include but are not limited to acetate, trifluoroacetate, sulfate, carbonate, halide, nitrate, perchlorate, propionate, pentafluoropropionate, acetylacetonate, and hexafluoroacetylacetonate, “m” is the oxidation state of M and “x” is the anion valence of X. The compounds described above are readily available from commercial suppliers, can be prepared by known methods or in certain cases can be prepared in situ by dissolving the corresponding metal oxide in the reaction solvent. For example, copper oxide can be dissolved in trifluoroacetic acid to provide copper trifluoroacetate.

Another component of the invention is a solvent in which the metal compounds described above are soluble. Non limiting examples of solvents are trifluoroacetic acid, trifluoroacetic anhydride, pentafluoropropionic acid, acetic acid, supercritical carbon dioxide and mixtures thereof with trifluoroacetic acid being preferred. The amount of compound which is added to the solvent can vary widely, but is usually from about 0.01 weight % to about 2 weight % of M as the metal. Another necessary ingredient of the process is an oxidant selected from the group consisting of hydrogen peroxide, organic hydroperoxides and mixtures thereof. Examples of organic hydroperoxides include but are not limited to tert-butylhydroperoxide, cumene hydroperoxide, etc. The amount of oxidant present in the solvent can also vary over a wide range, but is usually chosen to provide an oxidant to M molar ratio of about 15 to about 15000 and preferably from about 150 to about 1500. This mixture is now placed into a pressure vessel to which is added a methane stream in a concentration sufficient to produce a pressure of about 103 kPa (15 psi) to about 6895 kPa (1000 psi) and preferably from about 4137 kPa (600 psi) to about 6895 kPa (1000 psi). The pressurized reaction vessel is now heated at a temperature of about 25° C. to about 250° C. and preferably from about 60° C. to about 100° C. The vessel is held at this temperature for a time of about 30 minutes to about 24 hours in order to contact the methane with the oxidant, catalyst and solvent and provide a mixture comprising a methyl ester formed from the methane and an adduct from the solvent. Additional oxidant can be periodically added, i.e. intermittent addition, to obtain higher conversion of methane to the methyl ester.

The methyl ester formed, such as methyl trifluoroacetate, can be separated from the reaction mixture by any suitable methods but distillation is preferred. The methyl ester, e.g. methyl trifluoroacetate (MTFA) is now hydrolyzed to produce free methanol and regenerate the solvent. Using MTFA as an example, although it is understood that the process is not limited to MFTA, the MFTA is introduced into a hydrolysis reactor along with water. The amount of water introduced is at least the stoichiometric amount required for complete hydrolysis although it is preferred to use an excess amount of water. A catalyst and a co-solvent may also be used. A variety of acidic and basic substances are known to promote ester hydrolysis. Suitable acids include but are not limited to hydrochloric acid, sulfuric acid, trifluoroacetic acid, toluene sulfonic acid, acidic alumina, silica-alumina, sulfated zirconia, and acidic ion exchange resins. Suitable basic materials include but are not limited to sodium hydroxide, lithium hydroxide, potassium hydroxide, and solid bases such as hydrotalcite. Acid hydrolysis is preferred to allow easy recovery of the trifluoroacetic acid solvent/product. When hydrolysis is complete the methanol product can be separated from the reaction mixture by a variety of methods known in the art including distillation, adsorption, extraction and diffusion through a membrane. Separation of trifluoroacetic acid is achieved by analogous methods. The recovered trifluoroacetic acid is then recycled to the oxidation reactor.

In addition to carrying out the process in a batch mode as described above, the process can also be conducted in a continuous mode as follows. Methane, oxidant, solvent and/or catalyst are introduced via a liquid phase pump to a stirred high pressure liquid reactor. Gas and liquid are removed from the reactor continuously at a rate to maintain the liquid level and total pressure in the reactor. The removed gas/liquid stream is transferred to a vessel where the gas and liquid are separated and one or both streams may be subjected to further separation or returned to the high pressure reactor.

The following examples are presented in illustration of this invention and are not intended as undue limitations on the generally broad scope of the invention as set out in the appended claims.

### EXAMPLE 1

To an 80 cc glass liner placed in a dry ice bath there were added 8 ml of trifluoroacetic anhydride with stirring. To the solution there were added 1.0 cc of a 36% H₂O₂ aqueous solution at a rate such that the solution temperature was below 20° C. The solution was transferred to an 80 cc Parr™ reactor containing 30 mg of Cu(OCOCF₃)₂. The reactor was assembled and pressurized with methane (mixture of 95% methane and 5% neon as internal standard) to 800 psi and then heated to 80° C. for 3 hours. After the reaction, the gas sample was analyzed by gas chromatography (GC), and the liquid sample analyzed by both GC and nuclear magnetic resonance (NMR) spectroscopy with propionic acid added as an internal standard. The percent yield was calculated based on methyl trifluoroaceate product isolated divided by oxidant and methane substrate introduced into the system and gave 16.5% oxidant based yield and 1.2% methane based yield.

### EXAMPLE 2

To an 80 cc glass liner placed in a dry ice bath there were added 8 ml of trifluoroacetic anhydride with stirring. To the solution there were added 1.0 cc of a 36% H₂O₂ aqueous solution at a rate such that the solution temperature was below 20° C. The solution was transferred to an 80 cc Parr reactor containing palladium acetylacetonate, Pd(acac)₂, (30 mg). The reactor was assembled and pressurized first with nitrogen to about 675 psi, followed by the addition of methane (mixture of 95% methane and 5% neon as internal standard) to bring the total pressure of the reactor to about 750 psi and then heated to 80° C. for 20 hours. After the reaction, the gas sample was analyzed by GC, and the liquid sample analyzed by both GC and NMR with propionic acid added as internal standard. The percent yield was calculated based on methyl trifluoroaceate product isolated divided by oxidants and methane substrate introduced into the system and gave 13% oxidant based yield and 9.5% methane based yield.

### EXAMPLE 3

To an 80 cc Parr reactor there were added 40.5 g of potassium persulfate 4.05 g and 30 mg copper acetate, followed by the addition of 15 ml trifluoroacetic acid and trifluoroacetic anhydride (3 ml). The reactor was then assembled and pressurized with nitrogen to 450 psi, followed by the addition of methane (mixture of 95% methane and 5% neon as internal standard) to bring the reactor pressure to 500 psi and then heated to 100° C. for 20 hours. After the reaction, the gas sample was analyzed by GC, and the liquid sample analyzed by both GC and NMR with propionic acid added as an internal standard. Percent yield was calculated based on methyl trifluoroaceate product isolated divided by oxidants and methane substrate introduced into the system and gave 11.4% oxidant based yield and 10.7% methane based yield.

### EXAMPLE 4

To an 80 cc glass liner placed in a dry ice bath there were added 8 ml of trifluoroacetic anhydride with stirring. To the solution there were added 1.0 cc of a 36% H₂O₂ aqueous solution at a rate such that the solution temperature was below 20° C. The solution was transferred to an 80 cc Parr reactor containing Pd(OCOCF₃)₂ (30 mg). The reactor was assembled and pressurized first with nitrogen to about 675 psi, followed by the addition of methane (mixture of 95% methane and 5% neon as internal standard) to bring the total pressure of the reactor to about 750 psi and then heated to 80° C. for 1 hour. After the reaction, the gas sample was analyzed by GC, and the liquid sample analyzed by both GC and NMR with propionic acid added as an internal standard. Percent yield was calculated based on methyl trifluoroaceate product isolated divided by oxidants and methane substrate introduced into the system and gave 10.6% oxidant based yield and 6.3% methane based yield was observed.

What is claimed is:

1. A process for converting methane to methanol comprising contacting a gas stream comprising methane with a homogeneous catalyst and an oxidant selected from the group consisting of hydrogen peroxide, organic hydroperoxide and mixtures thereof at oxidation conditions to provide a methyl-ester and hydrolyzing the methyl ester at hydrolysis conditions to provide a methanol product stream; the homogeneous catalyst comprising a metal compound, having the empirical formula MₓXₘ where M is selected from the group consisting of palladium, copper, manganese, mercury, silver, cobalt, vanadium, platinum, lead, gold, niobium, chromium, molybdenum, tungsten, cerium and mixtures thereof, X is an anion selected from the group consisting of acetate, trifluoroacetate, sulfate, carbonate, halide, nitrate, perchlorate, propionate, pentafluoropropionate, acetylacetonate, and hexafluoroacetylacetonate, “m” is the oxidation state of M, and “x” is the anion valence of X, the metal compound dissolved in a solvent selected from the group consisting of trifluoroacetic acid, trifluoroacetic anhydride, pentafluoropropionic acid, acetic acid, supercritical carbon dioxide and mixtures thereof, and the oxidation conditions comprise a temperature of about 25° C. to about 250° C., a pressure of about 103 kPa (15 psi) to about 6895 kPa (1000 psi), a contact time of about 30 min. to about 24 hrs and an oxidant: M molar ratio of about 15 to about 15000.

2. The process of claim 1 where the oxidant is intermittently added.

3. The process of claim 1 where M is palladium.

4. The process of claim 1 where the solvent comprises trifluoroacetic acid.

5. The process of claim 1 where the process is a batch process.

6. The process of claim 1 where the process is a continuous process.

7. The process of claim 1 where the oxidant is hydrogen peroxide.

8. The process of claim 3 where the metal compound is palladium trifluoroacetate.

9. The process of claim 1 where the hydrolysis conditions comprise a temperature of about 20° C. to about 200° C. and a pressure of about 103 kPa to about 1030 kPa.

10. The process of claim 1 where the metal compound is copper acetate.

11. The process of claim 1 where the metal compound is palladium acetylacetonate.
